(19) United States
(12) Patent Application Publication
(10) Pub. No.: US 2002/0120313 A1
(43) Pub. Date: Aug. 29, 2002

(54) Tanning cushion

(76) Inventor: Angela M. Grant, Cuyahoga Falls, OH (US)

Correspondence Address: Heather M. Barnes, Emerson & Skeriotis, Fourteenth Floor, One Cascade Plaza, Akron, OH, 44308, US

(21) Appl. No.: 09/887,634

(22) Filed: Jun. 22, 2001

Related U.S. Application Data
(60) Provisional application No. 60/271,298, filed on Feb. 23, 2001.

(51) Int. Cl. A61N 1/00

(52) U.S. Cl. 607/91; 607/94

(57) ABSTRACT

A tanning cushion is adapted to be used by a sunbather in a tanning bed. The tanning cushion comprises at least one inflatable panel. Each inflatable panel has a plurality of support chambers. The inflatable panel inflates up to three inches, and is made of a substantially transparent material adapted to permit ultra-violet radiation to pass therethrough. The inflatable panel has a sufficient number of support chambers to provide comfort during a tanning session and to prevent pressure points from forming on the sunbather. The tanning cushion further comprises inflation means for inflating the plurality of support chambers.

This utility patent application claims priority from a provisional application having serial No. 60/271,298, which was filed on Feb. 23, 2001.

### BACKGROUND OF THE INVENTION

1. Field of Invention

This invention pertains to the art of methods and apparatuses for a tanning cushion, and more specifically to methods and apparatuses for an inflatable cushion to be used in conjunction with a tanning bed.

2. Description of the Related Art

Tanning beds have become a popular alternative to traditional sunbathing. The convenience of tanning beds allows the consumer to have flexibility as to when and where they would like to sunbathe. Further, the intensity of ultra-violet exposure received during one tanning session takes only a fraction of the time than trying to obtain the same effect by lying in the sun. Sunbathers can maintain a tan year round, even during the cold winter months.

Despite all the advantages and conveniences of tanning beds, several disadvantages still exist: sanitary conditions, comfort, and the formation of pressure points on the sunbather's body. It is well known that tanning salons have policies that the tanning beds are to be cleaned with a disinfectant cleaner once a tanning session is finished. Unfortunately, there is no sure way to guarantee that every sunbather adheres to these sanitation and cleaning policies. Even if consumers do clean the tanning bed after their session, it is very difficult to determine if the sunbather cleaned the tanning bed properly and thoroughly.

Another disadvantage of tanning beds is the lack of comfort. The lower tanning bed portion is usually made of Plexiglas, acrylic or other suitable material that permits ultraviolet light to pass through the Plexiglas to the sunbather's body. Since the Plexiglas is very hard, it is very uncomfortable. Those who use tanning beds usually prefer that their tanning bed session be a relaxing experience. It is difficult to relax when laying on such a hard surface.

Another problem encountered by many tanning bed users is pressure points forming on the shoulder blade and tailbone regions of the sunbather's body due to improper support on the tanning bed. These pressure points do not tan and remain the same color as the sunbather's natural skin pigmentation. In some cases, these pressure points appears as spots on the sunbather's body, which is undesirable.

Therefore, there is a need in the art for a tanning cushion, which provides comfort to the user, guarantees that the consumer will be using the tanning bed in a sanitary environment, and provides for an even suntan in the shoulder blade and tailbone regions.

### SUMMARY OF THE INVENTION

A tanning cushion is adapted to be used by a sunbather in a tanning bed. The tanning cushion comprises at least one inflatable panel. Each inflatable panel has a plurality of support chambers. The inflatable panel inflates up to three inches, and is made of a substantially transparent material adapted to permit ultra-violet radiation to pass therethrough. The inflatable panel has a sufficient number of support chambers to provide comfort during a tanning session and to prevent pressure points from forming on the sunbather. The tanning cushion further comprises inflation means for inflating the plurality of support chambers. Therefore, it is an object of the present invention to provide a tanning bed cushion, which provides comfort to the consumer.

It is yet another object of the present invention to provide a tanning bed cushion that prevents pressure points from forming on the shoulder blade and tailbone regions of the user.

Further, another object of the present invention is to provide a tanning bed cushion which permits ultraviolet radiation to pass therethrough.

Yet, another object of the present invention is to provide a tanning bed cushion, which provides sanitary tanning conditions for the sunbather.

Another object of the present invention is to provide a tanning bed cushion that is formed of three separate inflatable panels to provide easy portability.

Still yet, another object of the present invention is to provide a tanning bed cushion comprising an inflation height of no more than three inches.

Another object of the present invention is to provide a tanning bed cushion comprising an inflation height of no more than 1.5 inches.

Further, another object of the present invention is to provide a tanning bed cushion having a plurality of inflatable panels, where each of said inflatable panels comprises three columns and thirteen rows of support chambers.

Further yet, another object of the present invention is to provide a tanning bed cushion comprising three separate inflatable panels, wherein a seam interconnects each of said panels.

Still other benefits and advantages of the invention will become apparent to those skilled in the art to which it pertains upon a reading and understanding of the following detailed specification.

### BRIEF DESCRIPTION OF THE DRAWINGS

The invention may take physical form in certain parts and arrangement of parts, a preferred embodiment of which will be described in detail in this specification and illustrated in the accompanying drawings which form a part hereof and herein:

FIG. 1 is a plan view of the present invention;

FIG. 2 is a top view of the present invention with two inflatable panels positioned side by side;

FIG. 3 is a perspective view of the present invention with three inflatable panels positioned side by side;

FIG. 4 is a perspective view of the present invention illustrating the air valve of one of the inflatable panels;

FIG. 5 is a top view of a second embodiment of the present invention;

FIG. 6 is a side view of FIG. 5 taken along line 6-6.

FIG. 7 is a perspective view of the second embodiment of the present invention illustrating a one piece tanning cushion; and,

FIG. 8 is a top perspective view of the second embodiment of the present invention in a folded position.

### DESCRIPTION OF THE PREFERRED EMBODIMENT

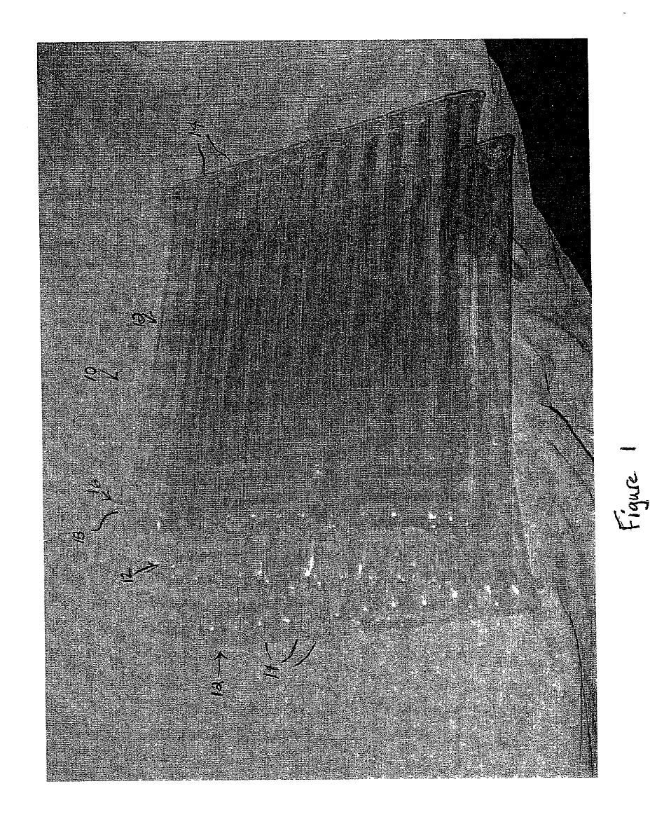

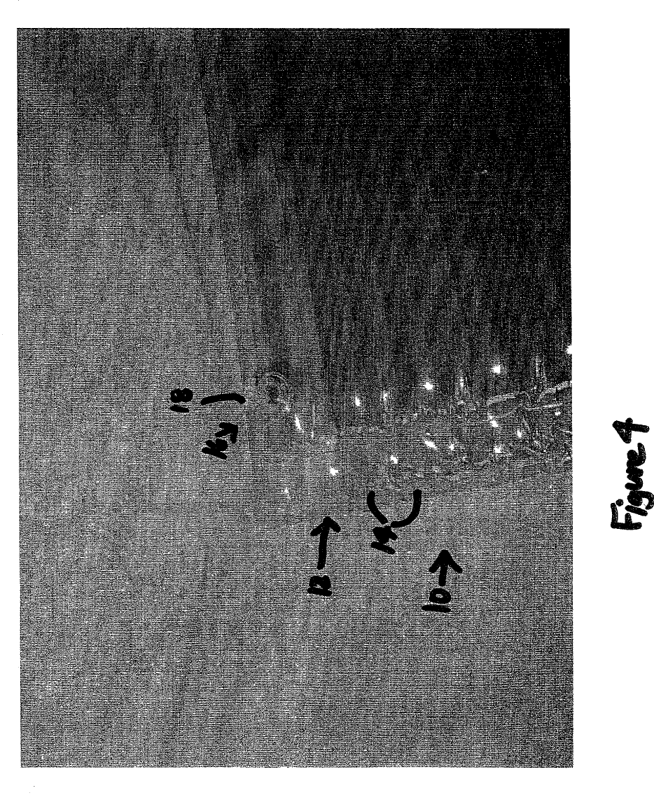

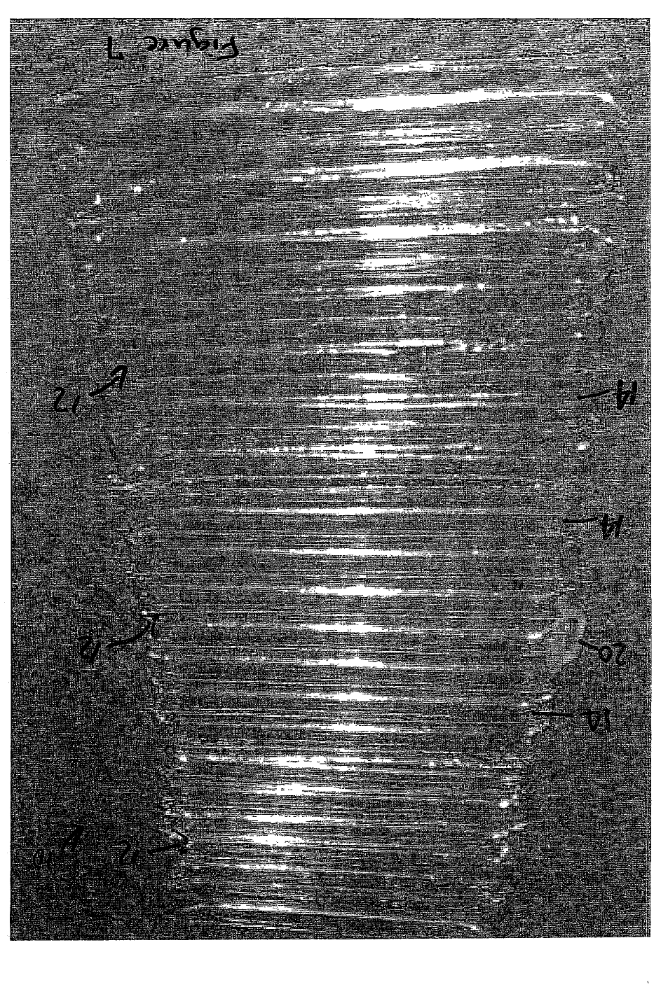

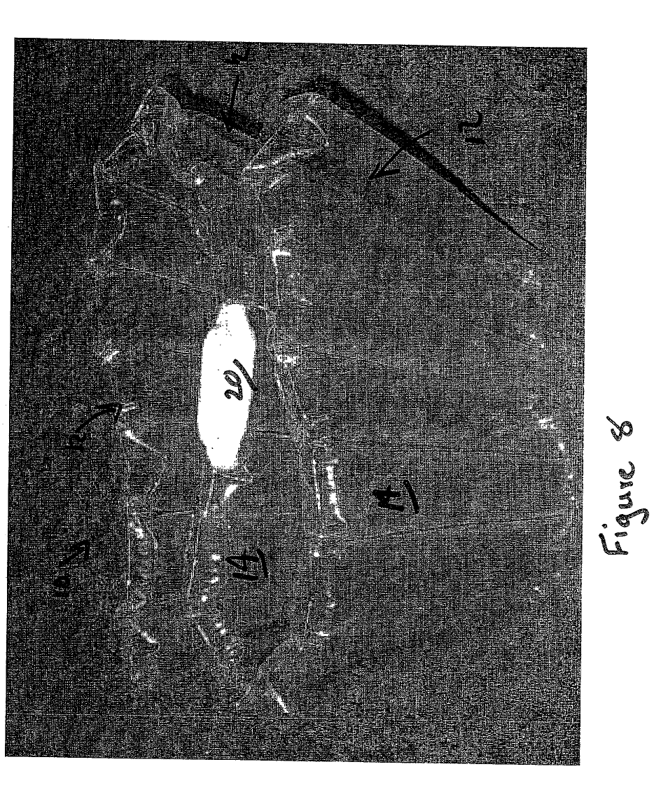

Referring now to the drawings wherein the showings are for purposes of illustrating a preferred embodiment of the invention only and not for purposes of limiting the same, FIGS. 1-8 illustrate the present invention. A tanning bed cushion 10 comprises a plurality of inflatable panels 12. Each of the inflatable panels 12 comprises a plurality of support chambers 14 and inflation means 16 for inflating the plurality of inflatable panels 12. The inflation means 16 may be an air valve 18 or other similar inflation device. The plurality of support chambers 14 is constructed so that the air passing through the air valve 18 inflates all the support chambers 14 of one inflatable panel 12.

Any number of support chambers 14 may be used; however, it is preferable that the number of support chambers 14 range from approximately ten to fifty support chambers 14 per inflatable panel 12. More preferably, there are approximately twenty-five to forty-five support chambers 14, and most preferably there are thirty-nine support chambers 14 arranged in three columns with thirteen rows. It is believed this provides the most support to the sunbather and prevents pressure points from forming on the sunbather's body. Of course, any number of support chambers 14 may be utilized provided the sunbather is properly and comfortably supported in the tanning bed. For example, in the second embodiment described below, the tanning cushion comprises approximately seven support chambers 14 per inflatable panel 12. As shown in FIGS. 1-4, the plurality of support chambers 14 is generally rectangular in shape. Any geometry may be used for the support chambers 14, including without limitation, squares or circles. The preferred embodiment utilizes a rectangular shape in order to accommodate as many support chambers 14 as possible on each inflatable panel 12.

So that the ultraviolet radiation produced by the bulbs of the lower tanning bed portion (not shown) is not compromised, the tanning bed cushion 10 should be constructed of a material that does not contain ultraviolet radiation inhibitors. Although any material of any thickness that permits ultraviolet radiation to pass therethrough may be utilized, the preferred material is a poly vinyl chloride (PVC) material. For example, such material may comprise about 65% PVC resin, about 32% plasticizer, about 1.5% stabilizer, and about 1.2% pigment, and is available through Middle Kingdom Inflatable World, Ltd. of Hong Kong, China, United Kingdom, and Spain. These percentages are not intended to be constructed as limitations, and for example only. The PVC material may have different percentages than those listed herein, as long as the PVC material permits ultra violet radiation to pass therethrough. The thickness of the PVC material may range from approximately six mills to eight mills. The thickness of the PVC material may also range from about 0.2 to about 0.4 millimeters. This thickness allows for easy storage of the tanning bed cushion 10. Further, the tanning bed cushion 10 material should be impervious to gas.

The inflatable panels 12 also comprise an inflation height H. It is preferable for the inflation height H to be no more than approximately three inches. As shown in FIGS. 1-8, the inflation height H should be no more than approximately three inches. In the preferred embodiment, the inflation height H should be no more than 1½ inches for the multi-piece tanning cushion shown in FIGS. 1-4, and it should be no more than approximately 2½ inches in the one piece tanning cushion 10 described below and shown in FIGS. 5-8. With a small inflation height, the sunbather remains close the lower tanning bed portion. This is important because some sunbathers may find tanning beds slightly claustrophobic. Given that the confined space is already small, a small inflation height H of the inflatable panels 12 does not compromise the amount of space between the lower tanning bed portion and the upper tanning bed portion. Further, a small inflation height H allows for the inflatable panels 12 to be easily rolled into a stored position upon completion of the tanning session. This is extremely convenient for the sunbather because tanning salons are usually very stringent about the allotted times each customer has for their appointment. With the present invention, the individual panels can be rolled and placed in a tote bag quickly and easily. There is no need to deflate the inflatable panels 12 since they have a relatively small inflation height H. It is within the scope of this invention that all three inflatable panels 12 may be rolled up together or individually before storing them. A strap with a handle may be tied around the rolled up inflatable panels 12. The strap may be Velcro, an elastic band, or a string tie. Alternatively, a handle 20 may be integrally formed with one or more of the inflatable panels 12 for easy carrying.

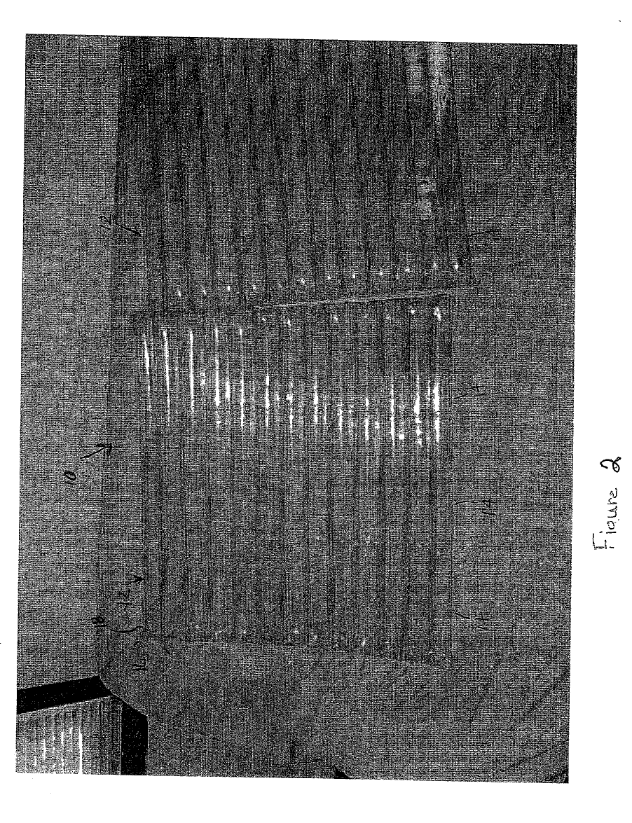

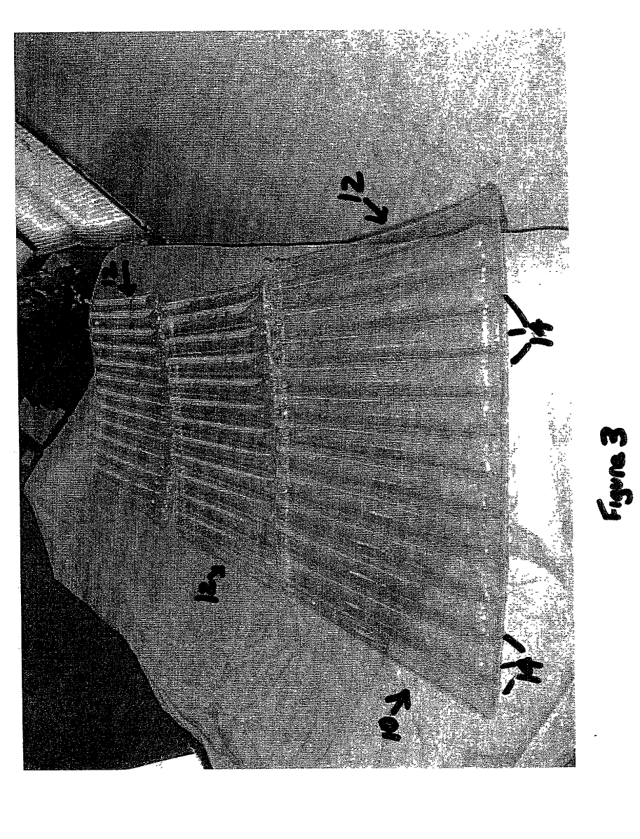

Although any number of inflatable panels 12 may be used, in one embodiment it is preferable to utilize multiple panels 12, such as three panels, as shown in FIG. 3. The inflatable panels 12 may be positioned in any desired location. In the preferred embodiment, three panels are aligned lengthwise to extend across the entire length of the lower tanning bed portion. Usually, this dimension may be two feet in width and six feet in length, but it is not limited thereto. Some sunbathers may prefer to eliminate the inflatable panel 12 that would normally be positioned under their head so that the neck region receives uniform tanning. Other people may be short and would only need two inflatable panels 12 to adequately support their body, as shown in FIG. 2. Thus, with the present invention, the inflatable panels 12 may be removed, added or repositioned to accommodate the sunbather's preferences.

When the inflatable panels 12 are positioned on the lower tanning bed portion, the inflatable panels 12 “stick” to the lower tanning bed portion and do not move. It is believed that enough friction and/or static electricity is present between the PVC material and the lower tanning bed portion to prevent the inflatable panels 12 from slipping out of position.

It is believed that the combination of the plurality of support chambers 14 and the low inflation height H results in the elimination of discomfort and pressure points on the sunbather. Pressure points, which result in spots in the shoulder blade and/or tailbone regions, are no longer a problem since the sunbather is properly supported. Because the sunbather lies on top of the inflatable panels 12 during the tanning session, sanitation of the lower tanning bed portion is not such a high concern. Ideally, the sunbather's body would not be directly contacting the lower tanning bed portion, although there may be minimal contact.

FIGS. 5-8 illustrate another embodiment of the present invention. In this embodiment, the tanning cushion 10 is one integral piece. Adjacent inflatable panels 12 are interconnected through a seam 22. The seam 22 comprises a single layer of material that also permits ultra-violet rays to pass therethrough. The seam material may be made of the same material as the inflatable panels 12. Further, the number of support chambers per inflatable panel may range from three to ten, but is preferably seven. This number of support chamber provides adequate support for the sunbather. The width of the tanning bed cushion 10 may range from substantially 25 inches to substantially 30 inches, but is most preferably 28 inches.

The tanning bed cushion 10 may also comprise handles 20 attached to the sides of each inflatable panel 12. Any number of handles 20 may be used, even one. Further, the handles 20 may be placed at any location, which makes transporting the tanning bed cushion 10 convenient for the consumer. In this embodiment, the tanning bed cushion 10 may be rolled into the stored position, or the tanning bed cushion 10 may be folded along the seams 22 for storage.

In order to use the present invention, each inflatable panel 12 is inflated through the inflation means 16. It may be inflated manually or through a small hand pump. If the user is not already at the location of the tanning bed, the user may roll up the tanning bed cushion 10 to transport it to the tanning bed. Once at the tanning bed, the user unrolls or unfolds the stored tanning bed cushion 10. If the multi-piece tanning cushion 10 is being utilized, the sunbather selectively positions the inflatable panels 12 as desired on the lower tanning bed portion. If the sunbather is utilizing the one piece tanning cushion 10, the tanning cushion 10 is simply positioned on the lower tanning bed portion. The sunbather then lies on top of the tanning bed cushion 10 throughout the tanning session and receives exposure to ultra-violet radiation. Once the tanning session is over, the user rolls or folds the tanning bed cushion 10 into a stored position, secures it with an elastic or Velcro strap, and leaves the tanning bed area. Of course it is recommended that the user still disinfect the lower tanning bed portion once the tanning session is finished. Using the present invention results in a comfortable and sanitary environment for the sunbather.

The invention has been described with reference to preferred embodiment. Obviously, modifications and alterations will occur to others upon a reading and understanding of this specification. It is intended to include all such modifications and alternations in so far as they come within the scope of the appended claims or the equivalence thereof.

Having thus described the invention, it is now claimed:

What is claimed is:

1. A tanning cushion adapted to be used by an associated sunbather in an associated tanning bed, the tanning cushion, comprising:
an inflatable panel comprising:
a plurality of support chambers; and,
an inflation height, said inflation height being up to approximately three inches, said inflatable panel being made of a substantially transparent material adapted to permit ultra-violet radiation to pass therethrough; and,
inflation means for inflating said plurality of support chambers.

2. The tanning cushion of claim 1, wherein said inflatable panel has a sufficient number of support chambers to prevent pressure points from forming on the associated sunbather.

3. The tanning cushion of claim 1, further comprising a plurality of inflatable panels.

4. The tanning cushion of claim 1, wherein said inflation height is no more than approximately two inches.

5. The tanning cushion of claim 1, wherein said inflation height is no more than approximately 1.5 inches.

6. The tanning cushion of claim 1, wherein said plurality of support chambers ranges from approximately twenty-five to approximately forty-five.

7. The tanning cushion of claim 6, wherein said plurality of support chambers are disposed in a plurality of rows and columns.

8. The tanning cushion of claim 7, wherein said plurality of support chambers are disposed in thirteen rows.

9. The tanning cushion of claim 7, wherein said plurality of support chambers are disposed in three columns.

10. The tanning cushion of claim 2, further comprising a seam, said seam interconnecting said plurality of support chambers.

11. The tanning cushion of claim 1, further comprising a handle attached to said inflatable panel.

12. The tanning cushion of claim 1, wherein said inflation means is an air valve.

13. A method for using a tanning cushion, comprising the steps of:
providing a tanning bed, said tanning bed having a lower tanning bed portion;
providing a tanning cushion, said tanning cushion comprising a plurality of inflatable panels, each of said inflatable panels having a plurality of support chambers, said plurality of inflatable panels having an inflation height up to approximately three inches, said plurality of inflatable panels being made of a substantially transparent material adapted to permit ultra-violet radiation to pass therethrough, said plurality of inflatable panels having a sufficient number of support chambers to prevent pressure points from forming on an associated sunbather, said tanning cushion comprising inflation means for inflating said plurality of inflatable panels;
inflating said inflatable panels with said inflation means;
positioning said tanning cushion on said underlying surface of said tanning bed;
laying on top of said tanning cushion; and,
receiving exposure from ultra-violet radiation.

14. The method of claim 13, further comprising the steps of:
ceasing exposure from ultra-violet radiation; and,
removing said tanning cushion from said tanning bed.

15. The method of claim 14, further comprising the step of:
rolling said tanning cushion into a stored position.

16. The method of claim 14, further comprising the step of:
folding said tanning cushion into a stored position.

17. A tanning system, comprising:
a tanning bed having a lower tanning bed portion, said lower tanning bed portion adapted to permit ultra-violet radiation to pass therethrough;
a tanning cushion adapted to be placed on said lower tanning bed portion, said tanning cushion, comprising:
at least one inflatable panel, said inflatable panel comprising a plurality of support chambers, said inflatable panel inflating up to three inches, said inflatable panel being made of a substantially transparent material adapted to permit ultra-violet radiation to pass therethrough, said inflatable panels having a sufficient number of support chambers to prevent pressure points from forming on the associated sunbather; and,
inflation means for inflating said inflatable panel.

18. The tanning system of claim 17, wherein said tanning cushion comprises three inflatable panels.

19. The tanning system of claim 18, wherein said lower tanning bed portion has a length and said three inflatable support panels are positioned to extend the length of said lower tanning bed portion.

20. The tanning system of claim 17, wherein said plurality of inflatable panels are interconnected through a seam.

21. The tanning system of claim 20, wherein said seam has a width, said width ranging from substantially two to substantially four inches.

22. The tanning system of claim 21, wherein said seam width is approximately three inches.

23. A tanning bed cushion adapted to be used by an associated sunbather in an associated tanning bed, said tanning bed cushion, comprising:
a plurality of inflatable panels having a plurality of support chambers;
a seam interconnecting adjacent inflatable panels, said seam being approximately three inches in width;
a handle attached to at least one of said plurality of inflatable panels; and,
inflation means for inflating said plurality of support chambers, said inflation means being a plurality of air valves, each of said air valves being operatively connected to each of said plurality of inflatable panels, said plurality of inflatable panels inflating no more than approximately 2.5 inches in height, said plurality of inflatable panels being made of a substantially transparent material adapted to permit ultra-violet radiation to pass therethrough, said plurality of inflatable panels providing sufficient support to prevent pressure points from forming on the associated sunbather.
